(19) United States
(12) Patent Application Publication
(10) Pub. No.: US 2010/0198348 A1
(43) Pub. Date: Aug. 5, 2010

(54) BIOMATERIALS WITH MODIFIED OPTICAL CHARACTER AND METHODS FOR PREPARING AND USING SAME

(76) Inventors: MICHAEL C. HILES, WEST LAFAYETTE, IN (US); DAVID A. ZOPF, ANN ARBOR, MI (US)

Correspondence Address: Woodard, Emhardt, Moriarty, McNett & Henry LLP, 111 Monument Circle, Suite 3700, Indianapolis, IN, 46204-5137, US

(21) Appl. No.: 12/694,653

(22) Filed: Jan. 27, 2010

Related U.S. Application Data
(60) Provisional application No. 61/148,713, filed on Jan. 30, 2009.

(51) Int. Cl. A61F 2/14

(52) U.S. Cl. 623/5.16

(57) ABSTRACT

Described are biocompatible materials treated with a biocompatible substance that embeds within pores of the materials so as to alter the transmittance of radiation through the materials. Remodelable materials such as collagenous ECM materials can be so treated to provide implants that are both remodelable and possess an increased capacity to transmit visible light and/or other forms of radiation. Such constructs find particular use in ophthalmic applications and, in particular cases, as corneal implant materials.

### REFERENCE TO RELATED APPLICATION

The present application claims the benefit of U.S. Provisional Patent Application Ser. No. 61/148,713 filed Jan. 30, 2009 entitled BIOMATERIALS WITH MODIFIED OPTICAL CHARACTER AND METHODS FOR PREPARING AND USING SAME which is hereby incorporated by reference in its entirety.

### TECHNICAL FIELD OF THE INVENTION

The present invention resides generally in the field of biomaterials and, more particularly, relates to biomaterials that have been modified to affect their optical character, including for example modifications to impart an increased capacity of the biomaterial to transmit radiative energy such as light.

### BACKGROUND OF THE INVENTION

Naturally derived and synthetic biomaterials have proven to be useful in a variety of applications, including for example cardiovascular surgery, orthopedic surgery, opthalmology, plastic surgery, urology, membranes for renal dialysis, and tissue regeneration. Several attempts have been made to increase the transparency of biomaterials while retaining their biocompatibility. For example, U.S. Pat. No. 4,505,855 discloses a transparent native, non-fibrilized collagen material having an absorbance at a wavelength of 900 nm of less than 5% in a sample 5 mm thick. The collagen material is made by centrifuging native soluble collagen to form a pellet and then fixing the pellet by either formaldehyde or glutaraldehyde or by irradiation to form crosslinks. The resulting material is said to be useful for a prosthetic replacement of the cornea.

U.S. Pat. No. 6,197,935 discloses collagen treated with heat, and by formic acid (FA), trifluoroacetic acid (TFA), tetrafluoroethanol (TFE) and hexafluoroisopropanol (HFIP) to produce a prion free collagen product for use as a biomaterial in a variety of applications, including a transparent material said to be useful as a corneal implant. In particular, this '935 patent teaches that prolonged treatment with TFA provides a transparent collagen, which transparency is further enhanced by adding glycosaminoglycans or proteoglycans, particularly hyaluronic acid.

U.S. Pat. No. 6,075,066 discloses semi-spherical materials to be worn on the eyeball, such as contact lenses for visual acuity correction or medical treatment use, cornea protecting materials, controlled drug release contact lenses and the like, which comprise, as a main component, a photocured, crosslinked glycosaminoglycan (e.g., hyaluronic acid and chondroitin sulfate). The crosslinking of the glycosaminoglycan is obtained by radiation-induced crosslinking of photoreactive groups covalently bonded to the glycosaminoglycan.

In view of the background in this area, there remain needs for improved and alternative biomaterials that have modified optical character, such as increased capacity to transmit radiative energy such as light. The present invention is addressed to these needs.

### SUMMARY OF THE INVENTION

In one embodiment, the present invention provides a medical implant comprising a porous biocompatible material and a biocompatible substance such as a gel embedded in the pores of the material and effective to increase the capacity of the material to transmit radiative energy such as light. In certain embodiments, the material is modified by the embedded substance so as to increase the capacity of the material to transmit light in the visible spectrum, for example to increase the relative transparency of the material. In certain embodiments, the biocompatible material is a remodelable material, such as a remodelable extracellular matrix material, e.g. a remodelable submucosa material.

In another embodiment, the present invention provides a method of increasing the capacity of a porous biocompatible material to transmit radiative energy. The method comprises treating the biocompatible material with a biocompatible substance so as to embed the substance in pores of the material and modify the capacity of the material to transmit radiative energy such as light. In certain embodiments, the material is modified by the embedded substance so as to increase the capacity of the material to transmit light in the visible spectrum, for example to increase the transparency of the material. In certain embodiments, the biocompatible material is a remodelable material, such as a remodelable extracellular matrix material, e.g. a remodelable submucosa material.

In another embodiment, the present invention provides a method of treating a damaged or diseased eye in a mammal. The method comprises providing a porous biocompatible material having a biocompatible substance embedded in pores of the material and effective to increase the capacity of the material to transmit visible light. This porous biocompatible material is implanted in the eye. In certain embodiments, the porous biocompatible material is implanted in contact with corneal tissue of the eye. Illustratively, the biocompatible material can be implanted in or on the cornea of the eye, or as a replacement of corneal tissue of the eye. Such corneal treatments can in certain embodiments involve the removal of at least a portion of the native cornea of the eye.

Additional embodiments as well as features and advantages of the invention will be apparent to those skilled in the art from the descriptions herein.

### BRIEF DESCRIPTION OF THE FIGURES

FIG. 1 provides a perspective view of a convexo-concave medical implant in accordance with the present invention.

FIG. 2 provides a perspective view of a medical implant in accordance with the present invention having an interior region modified to increase its transparency to light.

FIG. 3 provides a perspective view of a medical implant in accordance with the invention having a peripheral region modified to increase its transparency to light.

### DETAILED DESCRIPTION

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated device, and further applications of the principles of the invention as illustrated therein are herein contemplated as would normally occur to one skilled in the art to which the invention relates.

As disclosed hereinabove, in one aspect, the present invention provides a porous biocompatible material that has been treated with a biocompatible substance that embeds within pores of the biocompatible material and modifies the optical character of the material, for example by modifying the capacity of the material to pass or transmit electromagnetic radiation such as light. The present invention also provides related methods for preparing the treated biocompatible materials, and for using the materials for example in the treatment of patients or in scientific research.

The porous biocompatible material used in the present invention can be any of a wide variety of suitable materials known in the art. In certain embodiments of the invention, the biocompatible material is bioresorbable, for example as in the cases of bioresorbable naturally-occurring polymers and bioresorbable synthetic polymers. In desirable embodiments of the invention, the biocompatible material is a remodelable material, especially a remodelable collagen-containing material.

Remodelable collagenous materials when used in the invention can be comprised of a naturally-derived or reconstituted collagenous material, and in advantageous embodiments it is a collagen-containing extracellular matrix material (ECM). Collagenous extracellular matrix materials (ECMs) possessing biotropic properties, including in certain forms angiogenic collagenous extracellular matrix materials, can be used. Suitable extracellular matrix materials include, for instance, submucosa (including for example small intestinal submucosa, stomach submucosa, urinary bladder submucosa, or uterine submucosa), renal capsule membrane, dura mater, pericardium, serosa, peritoneum, amniotic membrane, or basement membrane materials, including liver basement membrane. These layers may be isolated and used as intact natural sheet forms, or reconstituted collagen layers including collagen derived from these materials or other collagenous materials may be used. For additional information as to submucosa materials useful in the present invention, and their isolation and treatment, reference can be made to U.S. Pat. Nos. 4,902,508, 5,554,389, 5,993,844, 6,206,931, and 6,099,567. Renal capsule tissue can also be obtained from warm blooded vertebrates, as described more particularly in copending U.S. patent application Ser. No. 10/186,150 filed Jun. 28, 2002, published Jan. 16, 2003 as U.S. Patent Application No. 20030014126) and International Patent Application serial No. PCT/US02/20499 filed Jun. 28, 2002, published Jan. 9, 2003 as W003002165.

Preferred porous biocompatible materials used in the invention will include an extracellular matrix material, such as submucosa, derived from a warm-blooded vertebrate. Mammalian submucosa or other extracellular matrix materials retaining substantially their native cross-linking are more preferred, although additionally crosslinked materials may also be used. In particular, extracellular matrix materials derived from animals raised for meat or other product production, e.g. pigs, cattle or sheep, will be advantageous. When used, submucosa can be derived from any suitable organ or other biological structure, including for example submucosa derived from the alimentary, respiratory, intestinal, urinary or genital tracts of warm-blooded vertebrates. Submucosa useful in the present invention can be obtained by harvesting such tissue sources and delaminating the submucosa from smooth muscle layers, mucosal layers, and/or other layers occurring in the tissue source. An ECM material that includes porcine-derived small intestinal submucosa provides a particularly preferred material for use in the present invention.

As prepared, an extracellular matrix (ECM) material for use in the present invention may optionally retain growth factors and/or other bioactive components native to the source tissue. For example, the matrix material may include one or more growth factors such as basic fibroblast growth factor (FGF-2), transforming growth factor beta (TGF-beta), epidermal growth factor (EGF), and/or platelet derived growth factor (PDGF). As well, submucosa or other ECM material of the invention may include other biological materials such as heparin, heparin sulfate, hyaluronic acid, fibronectin and the like. Thus, generally speaking, the ECM material may include a bioactive component that induces, directly or indirectly, a cellular response such as a change in cell morphology, proliferation, growth, protein or gene expression. Further, in addition or as an alternative to the inclusion of such native bioactive components, non-native bioactive components such as those synthetically produced by recombinant technology or other methods may be incorporated into the ECM material.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with a device including the material. In this regard, angiogenesis is the process through which the body makes new blood vessels to generate increased blood supply to tissues. Thus, angiogenic materials, when contacted with host tissues, promote or encourage the formation of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics, thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient.

A non-native bioactive component can be applied to a collagenous extracellular matrix material by any suitable means. Suitable means include, for example, spraying, impregnating, dipping, etc. The non-native bioactive agent can be applied to the collagenous extracellular matrix material either before or after the material is affixed to an elongate member. Similarly, if other chemical or biological components are included in the collagenous extracellular matrix material, the non-native bioactive component can be applied either before, in conjunction with, or after these other components.

ECM material used in the invention is preferably highly purified, for example, as described in U.S. Pat. No. 6,206,931. Thus, preferred material will exhibit an endotoxin level of less than about 12 endotoxin units (EU) per gram, more preferably less than about 5 EU per gram, and most preferably less than about 1 EU per gram. The ECM material may also have a bioburden of less than about 1 colony forming units (CFU) per gram, more preferably less than about 0.5 CFU per gram. Fungus levels are desirably low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plate forming units (PFU) per gram, more preferably less than about 5 PFU per gram.

As disclosed above, a biocompatible substance is embedded in pores of the porous biocompatible material so as to affect the optical character of the material. The biocompatible substance can be one or more of a variety of known biocompatible substances having the capacity to reside and be retained within the pores of the porous biocompatible material. Biocompatible gels or viscous liquids are advantageous for these purposes and include, for example, such materials containing naturally-occurring or synthetic materials. The embedded substance may for example include gelatin, collagen, one or more sugars such as sucrose, a polyhydric alcohol such as a glycol (e.g. polyethylene glycol) or glycerol, carboxymethyl cellulose, silicon, alginate, and the like, as well as mixtures including two or more of such materials. It will be understood that these are exemplary substances and that others may also be used within the scope of the present invention.

The biocompatible gel or other substance to be embedded in the material can be added in any suitable amount that imparts the desired modification to the reaction of radiation impinging upon the material. In certain embodiments, a biocompatible gel or other substance is added so as to substantially fill the pores of the biocompatible material in at least one region of the biocompatible material and potentially over the entirety of the biocompatible material. In certain embodiments, the biocompatible gel may occupy just the pores, with substantially no surface layer of the gel residing overtop the biocompatible material. In other embodiments, the biocompatible gel may occupy the pores of the biocompatible material and may also provide a substantially continuous layer of biocompatible gel covering at least one side of the biocompatible material and potentially both sides of the biocompatible material. These and other embodiments in which the biocompatible substance is applied in a manner that alters the optical characteristics of the biocompatible material will be readily apparent to those of ordinary skill in the art from the descriptions provided herein.

In some embodiments of the invention, the embedded biocompatible substance can have an index of refraction that is relatively close to the index of refraction of the substance from which the porous biocompatible material is made. For example, the biocompatible substance can have an index of refraction that is within the range of about 75% to about 125% of that of the substance from which the porous biocompatible material is made, or within the range of about 90% to about 110%, or even within the range of about 95% to about 105%. In this manner, it is believed that relative “index matching” can occur, resulting in a decrease in scattering and an increase in the capacity of the biocompatible material to transmit light or other similar radiation. In embodiments in which a collagenous porous biocompatible material such as an extracellular matrix is used, a biocompatible gel embedded in the pores may have an index of refraction in the range of about 1.1 to about 1.7, or in the range of about 1.2 to about 1.6, or in the range of about 1.3 to about 1.5. In situations in which aqueous biocompatible gel substances are used, the above-noted indexes of refraction can reflect those of the gel when hydrated and/or those of the gel when dehydrated. If needed or desired, pigment compounds may be used in conjunction with a biocompatible substance to prepare an embeddable substance having a given index of refraction.

In certain aspects of the invention, the embedded biocompatible substance will increase the transmittance of the biocompatible material to light of at least one wavelength in the visible range (i.e. wavelengths of about 400 nanometers (nm) to about 700 nm). In some embodiments, the embedded biocompatible substance will increase the transmittance of the biocompatible material to light of wavelengths across this entire visible range, effectively rendering the material more transparent to the eye such that things behind the material can be more clearly seen. An increase in the transmittance to light of a given wavelength or wavelengths can be reflected by a decrease in the optical density of the material at the given wavelength or wavelengths (OD=log₁₀(1/T), where OD is Optical Density and T is transmittance). In certain aspects of the invention, the embedded substance will reduce that optical density of the biocompatible material at least one visible light wavelength by at least 20%. In desirable inventive embodiments, this reduction by at least 20% will occur as to light of wavelengths across the entire visible spectrum (about 400 nm to 700 nm). In still further embodiments, the embedded substance will reduce the optical density of the biocompatible material by at least about 50%, and even by about 80% or more in advantageous embodiments, at least one visible light wavelength and optionally across the entire visible light spectrum. Moreover, in certain embodiments, the optical density of a treated ECM material of the present invention (e.g. a single layer ECM sheet material) in a dried condition will be less than about 0.5 across the entire visible spectrum (400-700 nm), more preferably less than about 0.3, and even more preferably less than about 0.2.

The application of the biocompatible substance to the porous biocompatible material may be conducted in any suitable fashion. For example, the biocompatible substance may be caused to infiltrate the pores of the biocompatible material by diffusion, by forced measures such as pressure driven techniques, by manual working of the substance into the pores, or any other suitable technique. In certain embodiments, the biocompatible substance may exhibit a less viscous state and a more viscous state, with the transition from the less to more viscous state caused by any suitable means including for example variations in physical properties such as temperature or hydration, chemical properties such as pH, radiation, and the like. Illustratively, gelatin preparations (e.g. derived from human, bovine, porcine or other animal sources) can be caused to infiltrate the pores of the porous biocompatible material at a temperature at or above the gel point of the gelatin, and then allowed to cool to stabilize the gelatin preparation within the pores. Gelatins of various gel points can be used for these purposes. In certain embodiments, the gel point will be above the body temperature of a subject to receive an implant of the inventive materials (e.g. above the human body temperature of about 37° C.), such that when implanted the gelatin can persist in its gelled state and resist dissolution from the pores. In other embodiments, the gelatin may have a gel point below the body temperature of an implant recipient (e.g. below about 37° C.), such that the gelatin is dissolved from the pores of the material over time. Mixtures of gelatins of varied gel points may also be used to achieve a combination of properties including dissolution and persistence of amounts of the gel from and within the pores of the biocompatible material. It will be understood that similar considerations apply to temperature-dependent gels formed of materials other than gelatin.

Materials of the invention can be produced in any desired thickness. In exemplary embodiments, the inventive material will have a thickness ranging up to about 2000 microns. This includes, for example, materials having a thickness in the range of about 10 microns to about 2000 microns, more typically in the range of about 50 to about 1000 microns.

Materials of the invention can be provided in the desired thickness using a single layer of a biocompatible material, or using multiple layers of a biocompatible material. In certain embodiments of the invention, materials of the invention will be formed as multilaminate collagen constructs. For example, a plurality of (i.e. two or more) layers of collagenous material, for example submucosa-containing or other ECM material, can be bonded together to form a multilaminate structure. Illustratively, two, three, four, five, six, seven, or eight or more collagenous layers containing submucosal or other collagenous ECM materials can be bonded together to provide a multilaminate collagenous material. The layers of collagenous tissue can be bonded together in any suitable fashion, including dehydrothermal bonding under heated, non-heated or cooled (e.g. lyophilization) conditions, vacuum pressing, using adhesives, glues or other bonding agents, crosslinking with chemical agents or radiation (including UV radiation), or any combination of these with each other or other suitable methods. When preparing inventive materials using multilaminate constructs, the biocompatible substance can be embedded within the pores of the layers of biocompatible material before they are bonded to one another, after they are bonded to one another, or any combination thereof. Further, the induction of crosslinking in or between layers of collagenous biomaterial in a multilaminate construct may also serve to introduce crosslinking in embedded biocompatible substances susceptible thereto, including for example biopolymer embedding materials such as gelatin or collagen. This may in turn also function to stabilize the embedded substance within the pores of the biocompatible substance and prevent or slow the migration or resorption of the embedded substance from the pores when the inventive material is implanted or otherwise situated in an aqueous environment. It will be understood that if desired, a similar stabilization of the embedded substance by crosslinking could also be performed in single-layer materials of the present invention.

When used, chemical cross-linking agents may include materials such as glutaraldehyde, formaldehyde, epoxides, genipin or derivatives thereof, carbodiimide compounds, polyepoxide compounds, or other similar agents. Crosslinking can also be catalyzed by exposure to UV radiation, treatment with enzymes such as transglutaminase or lysyl oxidase, and by photocrosslinking.

Biocompatible materials of the invention have a variety of uses including both medical (including veterinary) uses and research uses. In the medical field, biocompatible materials of the invention can be used for medical devices to be implanted into or onto tissues of a patient. Such devices may benefit from having all or a portion of the implant exhibit increased transmittance to radiation. For example, in the treatment of the eye, the implant may demonstrate improved transmission of visible light and thus improve the ability of the patient to see through the implant when located over or in the position of the cornea. In still further embodiments when treating the eye, the embedded material may be pigmented to selectively decrease the passage of certain wavelengths or ranges of wavelengths to which the patient may be sensitive during recovery or otherwise (e.g. as in the case of a selective decrease in the passage of ultraviolet light). In still other medical applications, implants may be used in procedures in which the passage of radiation through the material will be beneficial. Illustratively, this may occur when using radiation-activated agents such as bonding agents in conjunction with the implant, e.g. to facilitate attachment of the implant to a tissue of the patient. In research applications, biocompatible materials of the invention can be used for cell culture (e.g. as culture plate inserts) and when so used can provide enhanced observation using light microscopic techniques.

As noted above, in certain embodiments, the biocompatible material of the invention will be configured or used for treatment of the eye of a subject. Exemplary embodiments will include the repair or replacement of corneal and/or conjunctiva tissue of the eye. For example, the biocompatible material may be implanted in the treatment of corneal epithelial defects such as corneal ulcers (breaks in the outer layer of the epithelium of the cornea) and/or for ocular surface reconstruction. Ocular surface reconstruction may for example be undertaken to treat patients with limbal deficiency associated with hypofunction or total loss of stem cells. Stem cell hypofunction may result from any of a variety of causes including aniridia (hereditary), keratitis associated with multiple endocrine deficiency (hereditary), neurotrophic keratopathy (neural or ischemic), chronic limbitis, peripheral corneal ulcerative keratitis, pterygium or pseudopterygium. Limbal deficiency associated with total loss of stem cell function may be associated with chemical or thermal injuries to the ocular surface, Stevens-Johnson syndrome, repeated surgeries or cryotherapies to the limbal region, contact-lens induced keratopathy or toxic effects from lens-cleaning solutions. Such ocular resurfacing treatments can optionally be conducted with autograft limbal transplantation.

Biomaterials of the invention may also be used in the manufacture of protective shields to be applied to the eye in conjunction with another surgery. In particular embodiments, such protective shields may be manufactured using collagenous biomaterials of the invention, including bioactive ECM materials such as submucosa. Such protective shields may be resorbable over time and in the case of bioactive materials of the invention they may at the same time promote the healing of underlying injured tissue.

Biomaterials of the invention may also be used in the replacement of all or a portion of the cornea of an eye. In such procedures, at least a portion of a damaged or diseased cornea of a subject is removed, and a biomaterial of the invention is implanted in its place. The implant can be attached to the eye in any suitable fashion including for instance using sutures. The subject may for example be a human or other mammal. Corneal implants incorporating a biomaterial of the present invention may be provided in a shape corresponding to all or a portion of a native cornea. As well, they may be provided as single-layer or multiple-layer materials to provide the desired thickness, as discussed above.

Generally, medical implants incorporating materials of the present invention can be provided in a variety of shapes, including planar (e.g. sheet-form) and non-planar shapes. Exemplary non-planar shape implants include implants configured to have a concave surface, e.g. to substantially correspond to a convex surface of eye tissue against which the implant will reside. Thus, certain implants of the invention will have a convexo-concave structure in their relaxed state, for example in the case of a parabolic shape or a segment of a sphere (e.g. hemisphere). Such an implant may in some cases serve as a lens.

Materials of the invention may also have the biocompatible substance embedded in pores of all of or less than all of (e.g. in one or more regions of) the associated porous biocompatible material. Embodiments wherein only one or more regions of the porous biocompatible material carry the embedded substance may be employed, for example, in instances in which only regional modification of the optical properties of the biomaterial are needed or desired. Illustratively, in certain embodiments, only an interior region spaced from the periphery of a piece of biocompatible material may carry the embedded substance and exhibit a decreased optical density or other desired modification. Such an implant may for example be positioned on the eye with the interior region of greater transmittance positioned in the location of the cornea. In other embodiments, a band of material around the entire periphery or only segments of the periphery may be treated with the embedded substance and exhibit the modified optical character. Such an implant may for example be used in conjunction with a UV or visible light-activated bonding agent applied to a back surface of the periphery implant and activated by passing the activating light through the treated portions of the implant, wherein the embedded substance enhances the passage of the activating light through the material and thus facilitates the formation of an effective bond. This bond may for example be used in the attachment of all or one or more portions of the implant periphery to tissues of the patient, e.g. in the case of soft tissue reinforcement.

Still further medical applications of the inventive material include, for example, wound healing applications, tissue regenerative applications, cardiovascular applications, orthopedic applications, urologic applications, etc. In each of these and other medical applications the modified character of the inventive material when impinged by electromagnetic radiation may provide benefits in observation by attending medical personnel, and/or may provide other functional benefits as discussed above.

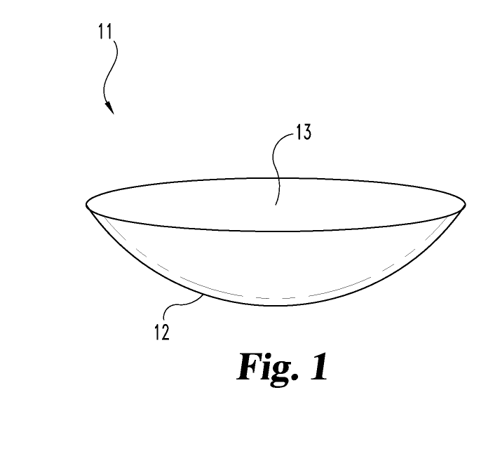

With reference now to FIGS. 1-3, shown are various implant configurations employing materials in accordance with the present invention. Particularly, shown in FIG. 1 is a medical implant 11 of the present invention having a generally convexo-concave shape, and thus possessing a convex surface 12 and an opposite concave surface 13. Implant 11 can, for example, be provided as an ocular implant, wherein concave surface 13 is configured to correspond to a naturally-occurring or surgically-created convex surface of the eye. Implant 11 may be treated in its entirety with an embedded substance that increases the transmittance of the biocompatible material to visible light, or may be treated over at least a portion of implant 11 that will reside over or in the location of the cornea of the eye.

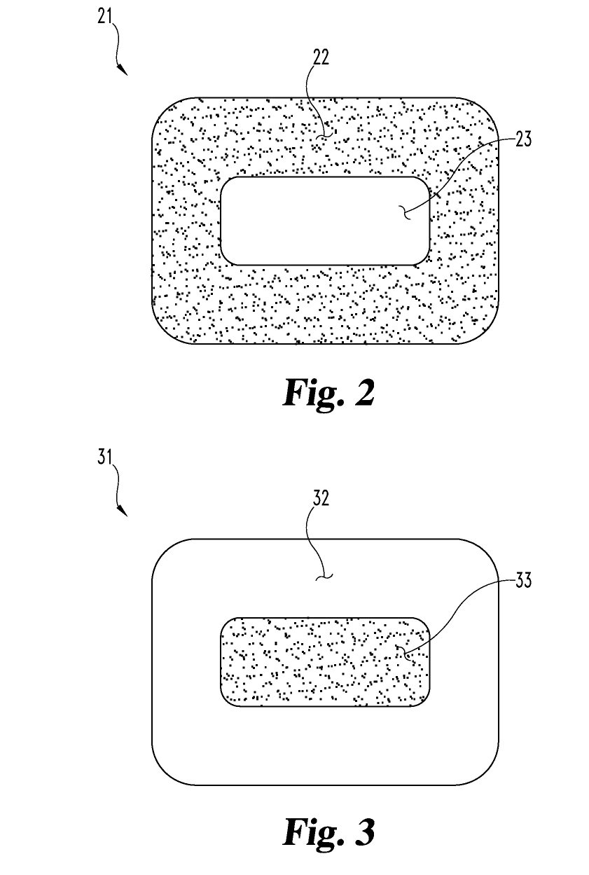

Shown in FIG. 2 is another implant 21 of the present invention. Implant 21 is a sheet-form implant that includes an internal region 22 having an embedded biocompatible substance in accordance with the present invention, and a peripheral region 23 surrounding the internal region that lacks the embedded biocompatible substance. In certain embodiments, the embedded substance will increase the transmittance of the internal region 22 to visible light relative to the peripheral region 23.

Referring now to FIG. 3, shown is an implant 31 of the present invention having a peripheral band 32 including the embedded biocompatible substance, and an internal region 33 lacking the embedded biocompatible substance. In certain embodiments, the embedded biocompatible substance can increase the transmittance of the peripheral band 32 to radiation such as ultraviolet or visible light. In exemplary modes of use, implant 31 may be employed with a radiation-activated bonding agent, wherein the activating radiation is passed through the implant 31 so as to contact and cure the bonding agent. The bonding agent may serve to bond the periphery of the implant 31 to tissues of a patient receiving the implant 31, for example in a medical application for soft tissue support.

Materials of the invention can be provided and packaged in a dehydrated or hydrated state. Dehydration of a medical material of the invention can be achieved by any means known in the art. For example, dehydration can be accomplished by lyophilization, including for instance freeze-drying or evaporative cooling techniques, air-drying, heating, or the like. When desired, a suitable aqueous medium can be used to rehydrate a dehydrated material of the invention prior to use. Illustratively, the aqueous medium can be pure water or a physiologically acceptable solution such as phosphate-buffered saline.

For the purpose of promoting a further understanding of the present invention, the following specific Example is provided. It will be understood that this Example is illustrative and not limiting of the invention.

### Example 1

A treatment solution was prepared by mixing deionized water, gelatin, sucrose, and neomycin sulfate, and heating the mixture to 50° C. to facilitate dissolution of all components. The solution contained 6.5% gelatin, 6.5% sucrose, and 1% neomycin sulfate. A lyophilized sheet of small intestinal submucosa (SIS) or renal capsule membrane (RCM) was rehydrated with deionized water and was flattened on a smooth surface. The treatment solution was cooled to about 37° C. and was distributed evenly over and diffused into the pores of the SIS or RCM material. The thus-treated SIS and RCM materials were then allowed to air dry under aseptic conditions.

Samples of the treated SIS and RCM materials were placed upon a 96-well microplate and subjected to a spectral scan at 1 nm wavelength increments over a range of 400 to 700 nm. The SIS and RCM samples included both dry samples and samples immersed in deionized water for 1, 10, and 60 minutes. In both their dry and hydrated forms, the treated SIS and RCM materials exhibited optical densities significantly lower than those of the corresponding untreated materials. Dry, treated SIS, RCM and other similar ECM materials (single layer) can be prepared to exhibit optical densities (OD's) across the full wavelength spectrum of 400 nm to 700 nm of less than about 0.3, or even less than about 0.2. Hydrated, treated SIS, RCM and other similar ECM materials (single layer) can be prepared to exhibit OD's across the full wavelength spectrum of 400 nm to 700 nm of less than about 0.8. The OD's of hydrated, treated ECM's vary with the soak time in water (starting with dried, treated material), with materials (including SIS and RCM) preparable to have OD's of less than about 0.6 across the full 400-700 nm spectrum after soaking for up to 10 minutes, and less than about 0.8 across the full 400-700 nm spectrum after soaking for up to an hour.

While the invention has been illustrated and described in detail in the drawings and foregoing description, the same is to be considered as illustrative and not limiting of the invention. In addition, all publications cited herein are hereby incorporated by reference as if each had been individually incorporated by reference and fully set forth.

What is claimed is:

1. A medical implant, comprising:
a porous biocompatible material;
a biocompatible substance embedded in pores of said porous biocompatible material; and
said embedded biocompatible substance effective to increase the transmittance of electromagnetic radiation through the porous biocompatible material.

2. The medical implant of claim 1, wherein the porous biocompatible material comprises collagen.

3. The medical implant of claim 2, wherein the porous biocompatible material comprises an extracellular matrix (ECM) material.

4. The medical implant of claim 1, wherein the embedded biocompatible substance is effective to decrease the optical density of the porous biocompatible material by at least about 20% in the wavelength range of 400 to 700 nm.

5. The medical implant of claim 1, wherein the index of refraction of the embedded biocompatible substance is about 90% to about 110% of the index of refraction of the substance from which the porous biocompatible material is made.

6. The medical implant of claim 1, wherein the embedded substance comprises gelatin or sucrose.

7. The medical implant of claim 3, wherein the ECM material comprises a material selected from the group consisting of pericardium, stomach submucosa, liver basement membrane, urinary bladder submucosa, dura mater, amniotic membrane, renal capsule membrane, and small intestinal submucosa (SIS).

8. The medical implant of claim 7, wherein the ECM material comprises SIS.

9. The medical implant of claim 1, which is configured for implantation in an eye.

10. The medical implant of claim 9, which is configured to replace all or a portion of a cornea of an eye.

11. A corneal implant, comprising:
a porous collagenous biomaterial configured for implantation in an eye to repair or replace a cornea of the eye; and
a biocompatible substance embedded within pores of the porous collagenous biomaterial;
said biocompatible substance effective to increase the transmittance of visible light through the porous collagenous biomaterial.

12. The corneal implant of claim 11, wherein the porous collagenous biomaterial comprises an extracellular matrix (ECM) material.

13. The corneal implant of claim 12, wherein the embedded biocompatible substance is effective to decrease the optical density of the porous collagenous biomaterial by at least about 20% in the wavelength range of 400 to 700 nm.

14. The corneal implant of claim 11, wherein:
the index of refraction of the embedded biocompatible substance is in the range of about 90% to about 110% of the index of refraction of the collagen of the porous collagenous biomaterial.

15. The corneal implant of claim 11, wherein the embedded biocompatible substance comprises gelatin.

16. The corneal implant of claim 12, wherein:
the ECM material comprises a material selected from the group consisting of pericardium, stomach submucosa, liver basement membrane, urinary bladder submucosa, dura mater, amniotic membrane, renal capsule membrane, and small intestinal submucosa (SIS).

17. The corneal implant of claim 16, wherein the ECM material comprises SIS.

18. The corneal implant of claim 16, wherein the biocompatible gel comprises gelatin.

19. The corneal implant of claim 16, wherein the biocompatible gel comprises collagen.

20. A method of modifying the capacity of a porous biocompatible material to transmit electromagnetic radiation, comprising treating the porous biocompatible material with a biocompatible substance that embeds in pores of the material and increases the capacity of the material to transmit electromagnetic radiation.

21. The method claim 20, wherein the biocompatible substance is effective to decrease the optical density of the porous biocompatible material by at least about 20% at least one wavelength in the range of about 400 to about 700 nm.

22. The method of claim 20, wherein the index of refraction of the biocompatible substance is in the range of about 90% to about 110% of that of the substance from which the porous biocompatible material is made.

23. The method of claim 20, wherein the embedded substance comprises gelatin.

24. The method claim 20, wherein the porous biocompatible material comprises a remodelable collagen-containing extracellular matrix (ECM) material.

25. The method of claim 24, wherein the ECM material comprises a member selected from the group consisting of pericardium, stomach submucosa, liver basement membrane, urinary bladder submucosa, dura mater, amniotic membrane, renal capsule membrane, and small intestinal submucosa (SIS).

26. The method of claim 25, wherein the ECM material comprises SIS.

27. The method of claim 20, wherein the porous biocompatible material is configured for implantation in an eye.

28. The method of claim 27, wherein the porous biocompatible material is configured for replacement or all or a portion of a cornea of the eye.

29. A method of treating a damaged or diseased cornea in a mammal, comprising:
(a) providing a remodelable collagenous material having pores, and a biocompatible substance embedded in said pores and effective to increase the capacity of the remodelable collagenous material to transmit visible light;
(b) removing at least a portion of the damaged or diseased cornea from the eye of the mammal, and
(c) implanting the remodelable collagenous material in the eye.

30. The method of claim 29, wherein the remodelable collagenous material is attached to the eye by suturing.

31. The method of claim 29, wherein the embedded biocompatible substance comprises gelatin.

32. The method of claim 29, wherein the remodelable collagenous material comprises an extracellular matrix (ECM) material.

33. The method of claim 32, wherein the ECM comprises a member selected from the group consisting of pericardium, stomach submucosa, liver basement membrane, urinary bladder submucosa, dura mater, amniotic membrane, renal capsule membrane, and small intestinal submucosa (SIS).

34. The method of claim 33, wherein the ECM comprises SIS.

35. The method of claim 29, wherein the embedded biocompatible substance is effective to decrease the optical density of the remodelable collagenous material by at least about 20% at least one wavelength in the range of 400 to 700 nm.

36. The method of claim 29, wherein the index of refraction of the embedded substance is in the range of about 90% to about 110% of that of the collagen of the remodelable collagenous material.
